(19) United States
(12) Patent Application Publication
(10) Pub. No.: US 2014/0046054 A1
(43) Pub. Date: Feb. 13, 2014

(54) PROCESS FOR PREPARING AMINES BY HOMOGENEOUSLY CATALYZED ALCOHOL AMINATION IN THE PRESENCE OF A COMPLEX CATALYST COMPRISING IRIDIUM AND AN AMINO ACID

(71) Applicant: BASF SE, Ludwigshafen (DE)

(72) Inventors: Alexander WETZEL, Heidelberg (DE); Michael Limbach, Worms (DE); Marion Brinks, Mannheim (DE); Mathias Schelwies, Heidelberg (DE)

(73) Assignee: BASF SE, Ludwigshafen (DE)

(21) Appl. No.: 13/961,345

(22) Filed: Aug. 7, 2013

Related U.S. Application Data
(60) Provisional application No. 61/681,154, filed on Aug. 9, 2012.

(51) Int. Cl. C07C 209/16; C07C 227/18; C07D 295/023; C07C 209/18

(52) U.S. Cl. CPC C07C 209/16; C07C 209/18; C07C 227/18; C07D 295/023
USPC 540/484; 564/480; 564/402; 560/19; 560/48; 546/184; 564/447; 548/578; 544/178

(57) ABSTRACT

The invention relates to a process for preparing amines (A) by alcohol amination of alcohols (Al) by means of an aminating agent (Am) with elimination of water, wherein the alcohol amination is carried out in the presence of a complex catalyst comprising iridium and an amino acid.

### DESCRIPTION

This patent application claims the benefit of pending U.S. provisional patent application Ser. No. 61/681,154 filed on Aug. 9, 2012, incorporated in its entirety herein by reference.

The present invention relates to a process for preparing amines (A) by homogeneously catalyzed alcohol amination of alcohols (Al) by means of an aminating agent (Am) with elimination of water.

Preferred amines (A) which can be prepared by the process of the invention are secondary or tertiary amines. Secondary amines (As) have at least one secondary amino group (>NH). Tertiary amines (At) have at least one tertiary amino group (>N—).

Amines (A) are valuable products having a large number of different uses, for example as solvents or stabilizers, for the synthesis of chelating agents, as starting materials for preparing synthetic resins, inhibitors, surface-active substances, as intermediates in the preparation of fuel additives, surfactants, drugs and crop protection agents, hardeners for epoxy resins, catalysts for polyurethanes, as intermediates for preparing quaternary ammonium compounds, plasticizers, corrosion inhibitors, synthetic resins, ion exchangers, textile assistants, dyes, vulcanization accelerators and/or emulsifiers.

The preparation of secondary and tertiary amines is described, for example, in K. Fujita, Z. Li, N. Ozeki, R. Yamaguchi, Tetrahedron Lett. 2003, 44, 2687-2690 and K. Fujita, Y. Enoki, R. Yamaguchi, Tetrahedron 2008, 64, 1943-1954. In the processes described there, [Cp*IrCl₂]₂ is used as catalyst in the presence of a base such as K₂CO₃ or NaHCO₃ and toluene as solvent. Primary or secondary alcohols can be used as starting materials for preparing the secondary or tertiary amines. Primary or secondary amine starting materials are used as amine component, with primary amine starting materials leading to secondary amines and secondary amine starting materials leading to tertiary amines. A disadvantage of the above-described processes is that the use of bases such as K₂CO₃ or NaHCO₃ is absolutely necessary. The bases used have to be separated off in an additional process step. In addition, temperatures of significantly above 100° C. are necessary to achieve acceptable yields. This makes the above-described processes energy-intensive and also unsuitable for thermally labile starting materials.

O. Saidi, A. J. Blacker, M. M. Farah, S. P. Marsden, J. M. J. Williams Chem. Commun. 2010, 46, 1541-1543 and R. Kawahara, K. Fujita, R. Yamaguchi, Adv. Synth. Catal. 2011, 353, 1161-1168 describe processes for preparing secondary or tertiary amines. [Cp*IrI₂]₂ or [Cp*Ir(NH₃)₃][α]₂, where “X” is Cl, Br or I, are used as catalysts. Here, primary or secondary alcohols are reacted with primary or secondary amine starting materials. The reaction is carried out in water, with the use of a base not being absolutely necessary. A disadvantage of these processes is that temperatures of significantly above 100° C. are likewise necessary to achieve acceptable yields. These processes, too, are therefore energy-intensive and have only limited suitability for the reaction of thermally labile starting materials.

Although the homogeneously catalyzed preparation of secondary and tertiary amines is described in the prior art, there is nevertheless a great need for alternative processes which display good activities and selectivities even at temperature below 100° C.

It is therefore an object of the present invention to provide a process for preparing amines, in particular secondary or tertiary amines, which gives the amines in good yields and selectivities and in which the formation of undesirable by-products is very largely avoided. In addition, the process should also be able to be carried out at low temperatures, preferably at temperatures below the temperatures of the processes described in the prior art.

The object is achieved by a process for preparing amines (A) by alcohol amination of alcohols (Al) by means of an aminating agent (Am) with elimination of water, wherein the alcohol amination is carried out in the presence of a complex catalyst comprising iridium and an amino acid.

It has surprisingly been found that the complex catalysts comprising iridium and an amino acid which are used in the process of the invention give secondary or tertiary amines sometimes in significantly improved yields and selectivities compared to the processes described in the prior art. In addition, the complex catalyst used in the process of the invention makes it possible to prepare secondary or tertiary amines at temperatures lower than those in the processes described in the prior art. Furthermore, the use of bases is not absolutely necessary in the process of the invention.

In homogeneously catalyzed alcohol amination, the hydroxyl groups (—OH) of the alcohol (Al) used are reacted with the amino group of the aminating agent (Am) used to form a secondary (>NH) or tertiary amino group (>N—), with one molecule of water being formed per hydroxyl group reacted. Primary or secondary amines can be used as aminating agent (Am).

### Starting Materials

In the process of the invention, alcohols (Al) and aminating agents (Am) are used as starting materials.

Suitable alcohols (Al) are compounds which comprise at least one hydroxyl group (hereinafter also referred to as OH group). The OH group can be in the form of a primary alcohol group (—CH₂—OH) or in the form of a secondary alcohol group (>CH—OH). Alcohols (Al) which have at least one primary alcohol group are preferred as starting materials.

Suitable alcohols (Al) are virtually all known alcohols which meet the abovementioned prerequisites. The alcohols can be linear, branched or cyclic. The alcohols can also bear substituents which are inert under the reaction conditions of the alcohol amination, for example alkyloxy, alkenyloxy, dialkylamino and halogens (F, Cl, Br, I). Preference is given to using monoalcohols, diols, triols or polyols as alcohols (Al). Monoalcohols have one OH group. Diols have two OH groups. Triols have three OH groups. Polyols have more than three OH groups.

Suitable alcohols (Al) are, for example, those of the general formula (XX):

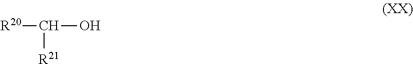

where
R²⁰ and R²¹ are selected independently from the group consisting of hydrogen, unsubstituted or at least monosubstituted C₅-C₁₀-cycloalkyl, C₅-C₁₀-heterocyclyl, C₅-C₁₄-aryl and C₅-C₁₄-heteroaryl or R²⁰ and R²¹ together with the carbon atom to which they are bound form a five- to fourteen-membered unsubstituted or at least monosubstituted ring system,
   where the substituents are selected from the group consisting of F, Cl, Br, OH, OR²², CN, NH₂, NHR²², N(R²²)₂, COOH, COOR²², C(O)NH₂, C(O)NHR²², C(O)N(R²²)₂, C₁-C₁₀-alkyl, C₅-C₁₀-cycloalkyl, C₅-C₁₀-heterocyclyl, C₅-C₁₄-aryl and C₅-C₁₄-heteroaryl,
   where R²² is selected from among C₁-C₁₀-alkyl and C₅-C₁₀-aryl.

When R²⁰ and R²¹ together with the carbon atom to which they are bound form a ring system, the ring system is preferably selected from the group consisting of unsubstituted or at least monosubstituted C₅-C₁₀-cycloalkyl, C₅-C₁₀-heterocyclyl, C₅-C₁₄-aryl and C₅-C₁₄-heteroaryl, where the substituents have the abovementioned meanings.

Particularly preferred ring systems are selected from the group consisting of unsubstituted or at least monosubstituted cyclopentyl, cyclohexyl, cycloheptyl, cyclooctyl, phenyl, naphthyl, anthryl and phenanthryl.

Examples of suitable monoalcohols are the following: methanol, ethanol, n-propanol, isopropanol n-butanol, 2-butanol, isobutanol, n-pentanol, ethanolamine (monoethanolamine), 2-ethylhexanol, cyclohexanol, benzyl alcohol, 2-phenylethanol, 2-(p-methoxyphenyl)ethanol, furfuryl alcohol, 2-(3,4-dimethoxyphenyl)ethanol, hydroxymethylfurfural, lactic acid, serine and fatty alcohols such as 1-heptanol (enanthic alcohol; C₇H₁₆O), 1-octanol (capryl alcohol; C₈H₁₈O), 1-nonanol (pelargonic alcohol; C₉H₂₀O), 1-decanol (capric alcohol; C₁₀H₂₂O), 1-undecanol (C₁₁H₂₄O), 10-undecen-1-ol (C₁₁H₂₂O), 1-dodecanol (lauryl alcohol; C₁₂H₂₆O), 1-tridecanol (C₁₃H₂₈O), 1-tetradecanol (myristyl alcohol; C₁₄H₃₀O), 1-pentadecanol (C₁₆H₃₂O), 1-hexadecanol (cetyl alcohol; C₁₆H₃₄O), 1-heptadecanol (C₁₇H₃₆O), 1-octadecanol (stearyl alcohol; C₁₈H₃₈O), 9-cis-octadecen-1-ol (oleyl alcohol; C₁₈H₃₆O), 9-trans-octadecen-1-ol (erucyl alcohol; C₁₈H₃₆O), all-cis-9,12-octadecadien-1-ol (linoleyl alcohol; C₁₈H₃₄O), all-cis-9,12,15-octadecatrien-1-ol (linolenyl alcohol; C₁₈H₃₂O), 1-nonadecanol (C₁₆H₄₀O), 1-eicosanol (arachidyl alcohol; C₂₀H₄₂O), 9-cis-eicosen-1-ol (gadoleyl alcohol; C₂₀H₄₀O), 5,8,11,14-eicosatetraen-1-ol (C₂₀H₃₄O), 1-heneicosanol (C₂₁H₄₄O), 1-docosanol (behenyl alcohol; C₂₂H₄₆O), 1-3cis-docosen-1-ol (erucyl alcohol; C₂₂H₄₄O) and 1-3trans-docosen-1-ol (brassidyl alcohol; C₂₂H₄₄O).

Particularly preferred monoalcohols are selected from the group consisting of methanol, ethanol, n-propanol, isopropanol, ethanolamine, 1-heptanol (enanthic alcohol; C₇H₁₆O), 1-octanol (capryl alcohol; C₈H₁₈O), 1-nonanol (pelargonic alcohol; C₉H₂₀O), 1-decanol (capric alcohol; C₁₀H₂₂O), 1-undecanol (C₁₁H₂₄O), 10-undecen-1-ol (C₁₁H₂₂O), 1-dodecanol (lauryl alcohol; C₁₂H₂₆O), 1-tridecanol (C₁₃H₂₈O), 1-tetradecanol (myristyl alcohol; C₁₄H₃₀O), 1-pentadecanol (C₁₈H₃₂O), 1-hexadecanol (cetyl alcohol; C₁₆H₃₄O), 1-heptadecanol (C₁₇H₃₆O), 1-octadecanol (stearyl alcohol; C₁₈H₃₈O), 9-cis-octadecen-1-ol (oleyl alcohol; C₁₈H₃₆O), 9-trans-octadecen-1-ol (erucyl alcohol; C₁₈H₃₆O), all-cis-9,12-octadecadien-1-ol (linoleyl alcohol; C₁₈H₃₄O), all-cis-9,12,15-octadecatrien-1-ol (linolenyl alcohol; C₁₈H₃₂O) and 1-nonadecanol (C₁₉H₄₀O).

The abovementioned fatty alcohols comprise both the pure compounds and also isomer mixtures of the primary fatty alcohols.

Ethanolamine can be used both as alcohol (Al) and as aminating agent (Am).

Examples of diols which can be used as starting materials in the process of the invention are 1,4-butanediol (1,4-butylene glycol), 1,5-pentanediol, 1,6-hexanediol, 1,7-heptanediol, 1,8-octanediol, 1,9-nonanediol, 9-cis-octadecene-1,12-diol (ricinoleyl alcohol; C₁₈H₃₆O₂), 2,4-dimethyl-2,5-hexanediol, the neopentyl glycol ester of hydroxypivalic acid, diethylene glycol, triethylene glycol, 1,4-bis(2-hydroxyethyl)piperazine, diisopropanolamine, N-butyldiethanolamine, 1,10-decanediol, 1,12-dodecanediol, 2,5-(dimethanol)furan, 1,4-bis(hydroxymethyl)cyclohexane and polyalkylene glycols whose OH groups can be either primary and/or secondary alcohols.

All known triols or polyols which have at least one functional group of the formula (—CH₂—OH) or (>CH—OH) can be used as starting materials. Examples of triols or polyols which can be used as starting materials in the process of the invention are glycerol, trimethylolpropane, triisopropanolamine, triethanolamine, polyvinyl alcohol, polyalkylene glycols whose OH groups can be either primary and/or secondary alcohols, 2,2-bis(hydroxymethyl)-1,3-propanediol (pentaerythritol), sorbitol, inositol, carbohydrates, sugars, sugar alcohols and sugar polymers: for example glucose, mannose, fructose, ribose, desoxyribose, galactose, N-acetylglucosamine, fucose, rhamnose, sucrose, lactose, cellobiose, maltose and amylose, cellulose, starch and xanthan.

As aminating agent (Am), it is possible to use virtually all known amines which have at least one primary or secondary amino groups.

Primary amines which are suitable as aminating agent (Am) are, for example, those of the general formula (XXI):

R³⁰NH₂ (XXI),

where
R³⁰ is selected from the group consisting of unsubstituted or at least monosubstituted C₁-C₃₀-alkyl, C₅-C₁₀-cycloalkyl, C₅-C₁₀-heterocyclyl, C₅-C₁₄-aryl and C₅-C₁₄-heteroaryl,
   where the substituents are selected from the group consisting of F, Cl, Br, OH, OR²², CN, NH₂, NHR²², N(R²²)₂, COOH, COOR²², C(O)NH₂, C(O)NHR²², C(O)N(R²²)₂, C₅-C₁₀-cycloalkyl, C₅-C₁₀-heterocyclyl, C₅-C₁₄-aryl and C₅-C₁₄-heteroaryl,
   where R²² is selected from among C₁-C₁₀-alkyl and C₅-C₁₀-aryl.

Primary amines suitable as aminating agent (Am) are, for example, the following: methylamine, ethylamine, n-propylamine, isopropylamine, n-butylamine, butan-2-amine, isobutylamine, tert-butylamine, n-pentylamine, n-hexylamine, 2-ethylhexylamine, aniline, cyclohexylamine, benzylamine, 2-phenylethylamine, 1-adamantylamine, 2-adamantylamine and fatty amines such as 1-heptanamine, 1-octanamine, 1-nonanamine, 1-decanamine, 1-undecanamine, 10-undecen-1-amine, 1-dodecanamine, 1-tridecanamin, 1-tetradecanamine, 1-pentadecanamine, 1-hexadecanamine, 1-heptadecanamine, 1-octadecanamine, 9-cis-octadecen-1-amine, 9-trans-octadecen-1-amine, 9-cis-octadecene-1,12-diamine, all-cis-9,12-octadecadien-1-amine; all-cis-9,12,15-octadecatrien-1-amine, 1-nonadecanamine, 1-eicosanamine, 9-cis-eicosen-1-amine, 5,8,11,14-eicosatetraen-1-amine, 1-heneicosanamine, 1-docosanamine, 1-3cis-docosen-1-amine and 1-3trans-docosen-1-amine.

The abovementioned fatty amines comprise the pure compounds and also isomer mixtures of the primary fatty amines.

Primary amines which are particularly preferred as aminating agent (Am) are selected from the group consisting of monomethylamine, 1-ethylamine, 1-propylamine, isopropylamine, aniline, ethanolamine and tert-butylamine.

Secondary amines suitable as aminating agent (Am) are, for example, those of the general formula (XXII):

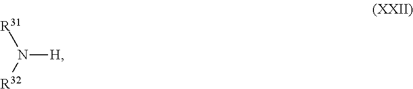

where
R³¹ and R³² are selected independently from the group consisting of unsubstituted or at least monosubstituted C₁-C₃₀-alkyl, C₅-C₁₀-cycloalkyl, C₅-C₁₀-heterocyclyl, C₅-C₁₄-aryl and C₅-C₁₄-heteroaryl or R³¹ and R³² together with the nitrogen atom to which they are bound form a five- to fourteen-membered unsubstituted or at least monosubstituted ring system,
   where the substituents are selected from the group consisting of F, Cl, Br, OH, OR²², CN, NH₂, NHR²², N(R²²)₂, COOH, COOR²², C(O)NH₂, C(O)NHR²², C(O)N(R²²)₂, C₁-C₁₀-alkyl, C₅-C₁₀-cycloalkyl, C₅-C₁₀-heterocyclyl, C₅-C₁₄-aryl and C₅-C₁₄-heteroaryl,
   where R²² is selected from among C₁-C₁₀-alkyl and C₅-C₁₀-aryl.

When R³¹ and R³² together with the nitrogen atom to which they are bound form a ring system, the ring system is preferably selected from the group consisting of unsubstituted or at least monosubstituted pyrrolidinyl, pyrrolyl, piperidinyl, where the substituents have the abovementioned meanings.

Secondary amines suitable as aminating agent (Am) are, for example, dimethylamine, diethylamine, diisopropylamine, di-n-propylamine, di-n-butylamine, dihexylamine, ditridecylamine, di-(2-ethylhexyl)amine, methylethylamine, piperidine, pyrrolidine, morpholine, N-methylaniline, dibenzylamine, tetrahydroquinoline.

Particularly preferred secondary amines are selected from the group consisting of dimethylamine and dibutylamine.

### Complex Catalyst

The process of the invention is carried out using at least one complex catalyst comprising the iridium as metal component and at least one amino acid as ligand. The complex catalyst preferably comprises an α-amino acid as ligand. The process of the invention is preferably carried out homogeneously catalyzed.

In a preferred embodiment, the process of the invention is carried out homogeneously catalyzed in the presence of a complex catalyst of the general formula (I):

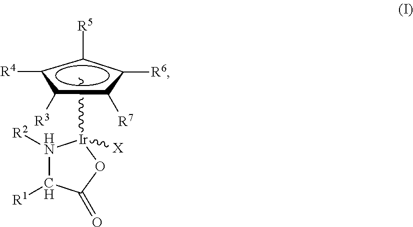

where
R¹ and R² are each, independently of one another, hydrogen, unsubstituted or at least monosubstituted C₁-C₁₀-alkyl, C₅-C₁₀-cycloalkyl, C₆-C₁₀-heterocyclyl, C₅-C₁₀-aryl or C₅-C₁₀-heteroaryl
   or
   R¹ and R² together with the atoms to which they are bound form an unsubstituted or at least monosubstituted five- to ten-membered ring system,
   where the substituents are selected from the group consisting of NR⁸R⁹, OR¹⁰, SR¹¹, C(O)OR¹², C(O)NR¹³R¹⁴, NHC(NH₂)₂⁺ and unsubstituted or at least monosubstituted C₅-C₁₀-aryl and C₆-C₁₀-heteroaryl,
   where the substituents are selected from among OH and NH₂;
X is fluoride, chloride, bromide or iodide;
R³, R⁴, R⁵, R⁶ and R⁷ are each, independently of one another, hydrogen, methyl, ethyl, n-propyl, isopropyl or phenyl;
R⁸, R⁹, R¹⁰, R¹¹, R¹², R¹³ and R¹⁴ are each, independently of one another, hydrogen or C₁-C₆-alkyl.

The complex catalyst can be uncharged or singly or doubly positively charged. The complex catalyst is preferably uncharged.

The substituent NHC(NH₂)₂⁺ is in the present case a substituent having the following structural formula, where the substituent is bound via the bond depicted as a broken line.

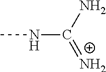

Particular preference is given to complex catalysts (I) in which R³, R⁴, R⁵, R⁶ and R⁷ are each methyl and X is chloride.

The complex catalyst of the general formula (I) has a stereo center on the central iridium atom. The general formula (I) comprises, according to the invention, all stereoisomers (in formula (I) depicted by the wavy bonds) and is not restricted to the configuration shown in formula (I). The formula (I) thus also comprises the stereoisomers (laa) and (Ibb).

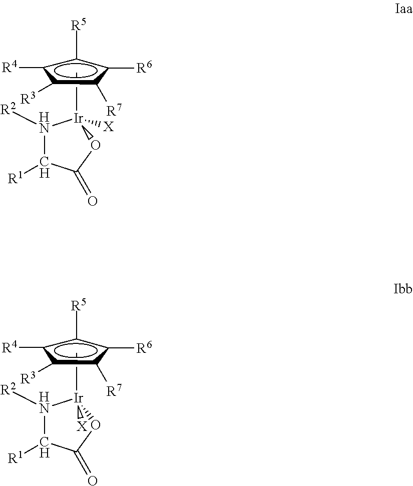

The same applies to the amino acids comprised as ligands in the complex catalyst (I). When the amino acids comprise one or more stereo centers, the complex catalyst (I) likewise comprises all stereoisomers. The invention also comprises derivatives of the complex catalyst (I) which can be obtained from the complex catalyst by protonation or deprotonation.

R. Krämer, K. Polborn, H. Wanjek, I. Zahn, W. Beck, Chem. Ber. 1990, 123, 767 describe the preparation and NMR-spetroscopic examination of various iridium complexes bearing Cp* (pentamethylcyclopentadienyl) and α-amino acids as ligands.

The complex catalysts (I) used according to the invention can be prepared from an iridium-comprising catalyst precursor by reaction with an amino acid in the presence of a solvent and a base.

Suitable iridium-comprising catalyst precursors, are, for example, [Cp*IrF₂]₂, [Cp*IrCl₂]₂, [Cp*IrBr₂]₂, [Cp*IrI₂]₂, with [Cp*IrCl₂]₂ being preferred. Suitable solvents for preparing the complex catalyst according to the invention are, for example, aprotic polar solvents, with acetonitrile being particularly preferred. Suitable bases are alkali metal or alkaline earth metal carbonates, with potassium carbonate (K₂CO₃) being preferred. The reaction is preferably carried out under a protective gas atmosphere, for example nitrogen or argon. The reaction temperature is generally from 0 to 100° C., preferably from 10 to 40° C. and particularly preferably from 15 to 25° C. The preparation of the complex catalyst (I) is preferably carried out at atmospheric pressure, i.e. ambient pressure.

The amino acid used as ligand is preferably used in equimolar amounts based on the iridium comprised in the iridium-comprising catalyst precursor.

The reaction time is in the range from 5 minutes to 100 hours, preferably in the range from 5 to 50 hours, preferably in the range from 15 to 30 hours.

To isolate the complex catalyst (I) according to the invention, the base used, preferably potassium carbonate, is generally filtered off. The solvent, preferably acetonitrile, is subsequently removed by distillation, optionally under reduced pressure. The complex catalyst (I) obtained in this way can, optionally after further work-up, be used as complex catalyst in the alcohol amination.

α-Amino acids are preferred as amino acids. The amino acids can be used both as L-α-amino acid and as D-α-amino acid. It is also possible to use mixtures of the abovementioned configurational isomers, known as D-L-α-amino acids.

As amino acids, it is possible to use both naturally occurring amino acids and also exclusively synthetic amino acids.

Preferred amino acids are selected from the group consisting of alanine, valine, leucine, isoleucine, proline, tryptophan, phenylalanine, methonine, glycine, serine, tyrosine, threonine, cysteine, asparagine, glutamine, aspartate, glutamate, lysine, arginine, histidine, citrulline, homocysteine, homoserine, (4R)-4-hydroxyproline, (5R)-5-hydroxylysine, ornithine and sarcosine. The abovementioned amino acids can be used both as L-α-amino acids and as D-α-amino acids. In addition, mixtures of L-α- and D-α-amino acids of the abovementioned amino acids can also be used.

Particularly preferred amino acids are selected from the group consisting of glycine, valine, proline and sarcosine. Very particularly preferred amino acids are selected from the group consisting of proline and sarcosine.

The above statements and preferences in respect of the amino acids apply analogously to the complex catalyst (I) containing the amino acid. The abovementioned statements and preferences in respect of the amino acids therefore likewise apply analogously to the ligands of the complex catalyst (I).

Preference is therefore given to complex catalysts (I) comprising iridium as metal component, Cp* (1,2,3,4,5-pentamethylcyclopentadienyl anion), chloride and an amino acid selected from the group consisting of alanine, valine, leucine, isoleucine, proline, tryptophan, phenylalanine, methonine, glycine, serine, tyrosine, threonine, cysteine, asparagine, glutamine, aspartate, glutamate, lysine, arginine, histidine, citrulline, homocysteine, homoserine, (4R)-4-hydroxyproline, (5R)-5-hydroxylysine, ornithine and sarcosine.

Particular preference is given to complex catalysts (I) comprising iridium as metal component, Cp* (1,2,3,4,5-pentamethylcyclopentadienyl anion), chloride and an amino acid selected from the group consisting of glycine, valine, proline and sarcosine. For the purposes of the present invention, C₁-C₃₀- or C₁-C₁₀-alkyl or C₁-C₆-alkyl are branched, unbranched, saturated and unsaturated groups. Preference is given to alkyl groups having 1 to 6 carbon atoms (C₁-C₆-alkyl). Greater preference is given to alkyl groups having from 1 to 4 carbon atoms (C₁-C₄-alkyl).

Examples of saturated alkyl groups are methyl, ethyl, n-propyl, isopropyl, n-butyl, isobutyl, sec-butyl, tert-butyl, amyl and hexyl.

Examples of unsaturated alkyl groups (alkenyl, alkynyl) are vinyl, allyl, butenyl, ethynyl and propynyl.

The C₁-C₁₀-alkyl group can be unsubstituted or substituted by one or more substituents selected from the group consisting of F, Cl, Br, hydroxy (OH), C₁-C₁₀-alkoxy, C₅-C₁₀-aryloxy, C₆-C₁₀-alkylaryloxy, C₅-C₁₀-heteroaryloxy comprising at least one heteroatom selected from among N, O, S, oxo, C₃-C₁₀-cycloalkyl, phenyl, C₅-C₁₀-heteroaryl comprising at least one heteroatom selected from among N, O, S, C₅-C₁₀-heterocyclyl comprising at least one heteroatom selected from among N, O, S, naphthyl, amino, C₁-C₁₀-alkylamino, C₆-C₁₀-arylamino, C₅-C₁₀-heteroarylamino comprising at least one heteroatom selected from among N, O, S, C₁-C₁₀-dialkylamino, C₁₀-C₁₂-diarylamino, C₁₀-C₂₀-alkylarylamino, C₁-C₁₀-acyl, C₁-C₁₀-acyloxy, NO₂, C₁-C₁₀-carboxy, carbamoyl, carboxamide, cyano, sulfonyl, sulfonylamino, sulfinyl, sulfinylamino, thiol, C₁-C₁₀-alkylthiol, C₅-C₁₀-arylthiol or C₁-C₁₀-alkylsulfonyl.

For the present purposes, the terms C₅-C₁₀-cycloalkyl refers to saturated, unsaturated monocyclic and polycyclic groups. Examples of C₅-C₁₀-cycloalkyl are cyclopentyl, cyclohexyl and cycloheptyl. The cycloalkyl groups can be unsubstituted or substituted by one or more substituents as defined above for the group C₁-C₁₀-alkyl.

For the purposes of the present invention, C₅-C₁₄-aryl or C₅-C₁₀-aryl is an aromatic ring system having from 5 to 14 or from 5 to 10 carbon atoms. The aromatic ring system can be monocyclic or bicyclic. Examples of aryl groups are phenyl, naphthyl such as 1-naphthyl and 2-naphthyl. The aryl group can be unsubstituted or substituted by one or more substituents as defined above under C₁-C₁₀-alkyl.

For the purposes of the present invention, C₅-C₁₄-heteroaryl or C₅-C₁₀-heteroaryl is a heteroaromatic system comprising at least one heteroatom selected from the group consisting of N, O and S. The heteroaryl groups can be monocyclic or bicyclic. When nitrogen is a ring atom, the present invention also comprises N-oxides of the nitrogen-heteroaryls. Examples of heteroaryls are thienyl, benzothienyl, 1-naphthothienyl, thianthrenyl, furyl, benzofuryl, pyrrolyl, imidazolyl, pyrazolyl, pyridyl, pyrazinyl, pyrimidinyl, pyridazinyl, indolyl, isoindolyl, indazolyl, purinyl, isoquinolinyl, quinolinyl, acridinyl, naphthyridinyl, quinoxalinyl, quinazolinyl, cinnolinyl, carbolinyl, thiazolyl, oxazolyl, isothiazolyl, isoxazolyl. The heteroaryl groups can be unsubstituted or substituted by one or more substituents defined above under C₁-C₁₀-alkyl.

For the purposes of the present invention, the term C₅-C₁₀-heterocyclyl refers to five- to ten-membered ring systems comprising at least one heteroatom from the group consisting of N, O and S. The ring systems can be mono or bicyclic. Examples of suitable heterocyclic ring systems are piperidinyl, pyrrolidinyl, pyrrolinyl, pyrazolinyl, pyrazolidinyl, morpholinyl, thiomorpholinyl, pyranyl, thiopyranyl, piperazinyl, indolinyl, dihydrofuranyl, tetrahydrofuranyl, dihydrothiophenyl, tetrahydrothiophenyl, dihydropyranyl and tetrahydropyranyl.

### Alcohol Amination

The complex catalyst (I) according to the invention is preferably used directly in its active form. For this purpose, the complex catalyst is prepared as described above in a process step preceding the actual alcohol amination. The alcohol amination is preferably carried out homogeneously catalyzed.

For the purposes of the present invention, homogeneously catalyzed means that the catalytically active part of the complex catalyst (I) is at least partly present in solution in the liquid reaction medium. In a preferred embodiment, at least 90% of the complex catalyst used in the process are present in solution in the liquid reaction medium, more preferably at least 95%, in particular more than 99% by weight, and the complex catalyst is most preferably entirely present in solution in the liquid reaction medium (100%), in each case based on the total amount in the liquid reaction medium.

The complex catalyst is used in amounts in the range from 0.01 to 20 mol %, preferably in the range from 0.1 to 10 mol % and particularly preferably in the range from 0.2 to 6 mol %, per mole of OH group comprised in the starting material for the alcohol amination.

The reaction is carried out in the liquid phase at a temperature of generally from 20 to 250° C. The process of the invention is preferably carried out at temperatures in the range from 50° C. to 150° C., particularly preferably in the range from 50 to 130° C. and in particular in the range from 70 to 99° C.

The liquid phase can be formed by the starting materials, i.e. the alcohol (Al) or the aminating agent (Am), and/or a solvent.

The reaction can generally be carried out at a total pressure of from 1 to 100 bar absolute, which can be both the intrinsic pressure of the solvent at the reaction temperature and the pressure of a gas such as nitrogen, argon or hydrogen. The process of the invention is preferably carried out at a total pressure in the range from 1 to 30 bar absolute, in particular at a total pressure in the range from 1 to 5 bar absolute.

In a preferred embodiment, the process of the invention is carried out in the absence of hydrogen. For the purposes of the invention, absence of hydrogen means that no additional hydrogen is introduced into the reaction. Any traces of hydrogen introduced via other gases and traces of hydrogen formed in the reaction are still counted as absence of hydrogen for the purposes of the present invention.

The average reaction time is generally from 15 minutes to 100 hours, preferably from 5 hours to 30 hours.

The aminating agent (Am) can be used in stoichiometric, substoichiometric or superstoichiometric amounts based on the hydroxyl groups to be aminated in the alcohol (Al). The aminating agent (Am) is preferably used in stoichiometric amounts.

The process of the invention can be carried out both in the presence of a solvent and without solvents. The process of the invention is preferably carried out in the presence of a solvent. Suitable solvents are polar and nonpolar solvents which can be used in pure form or in mixtures. For example, it is possible for only one nonpolar solvent or only one polar solvent to be used in the process of the invention. It is also possible to use mixtures of two or more polar solvents or mixtures of two or more nonpolar solvents or mixtures of one or more polar solvents with one or more nonpolar solvents.

Suitable nonpolar solvents are, for example, saturated and unsaturated hydrocarbons such as hexane, heptane, octane, cyclohexane, benzene, toluene, xylene (o-xylene, m-xylene, p-xylene) and mesitylene and linear and cyclic ethers such as diethyl ether, 1,4-dioxane, MTBE (tert-butyl methyl ether), diglyme and 1,2-dimethoxyethane. Preference is given to using toluene, xylenes or mesitylene. Particular preference is given to toluene.

Suitable polar solvents are, for example, water, dimethylformamide, formamide, tert-amyl alcohol and acetonitrile. Preference is given to using water. Water can be added before the reaction, be formed as water of reaction in the reaction or be added after the reaction in addition to the water of reaction.

The addition of bases can have a positive effect on product formation. Suitable bases which may be mentioned here are alkali metal hydroxides, alkaline earth metal hydroxides, alkali metal alkoxides, alkaline earth metal alkoxides, alkali metal carbonates, alkaline earth metal carbonates, alkali metal hydrogencarbonates and alkaline earth metal hydrogencarbonates, of which from 0.01 to 100 molar equivalents based on the metal catalyst used can be employed. However, the use of bases in the process of the invention is not absolutely necessary. In a preferred embodiment, the process of the invention is carried out without addition of the abovementioned bases.

In the case of the reaction in the liquid phase, the aminating agent (Am), the alcohol, preferably together with a solvent, and the complex catalyst (I) are introduced into a reactor.

The introduction of the aminating agent (Am), the alcohol (Al), the solvent and the complex catalyst (I) can be carried out simultaneously or separately. The reaction can be carried out continuously, in semibatch operation, in batch operation, backmixed in product as solvent or in a single pass without backmixing.

It is in principle possible to use all reactors which are fundamentally suitable for liquid reactions at the given temperature and the given pressure for the process of the invention. Suitable standard reactors for gas/liquid reaction systems and for liquid/liquid reaction systems are indicated, for example, in K. D. Henkel, “Reactor Types and Their Industrial Applications”, in Ullmann's Encyclopedia of Industrial Chemistry, 2005, Wiley-VCH Verlag GmbH & Co. KGaA, DOI: 10.1002/14356007.b04_{—}087, chapter 3.3 “Reactors for gas-liquid reactions”. Examples which may be mentioned are stirred tank reactors, tube reactors and bubble column reactors.

In the amination reaction, at least one primary or secondary hydroxyl group of the alcohols (Al) is reacted with the amino group of the aminating agent (Am) to form a secondary or tertiary amine, with in each case one mole of water of reaction being formed per mole of hydroxyl group reacted.

Secondary or tertiary amines can be obtained by the process of the invention.

Secondary amines (As) are obtained when a primary or secondary monoalcohol is used as alcohol (Al) and a primary amine is used as aminating agent (Am). Preference is given to using a primary alcohol (R²⁰ or R²¹ in formula (XX) is hydrogen) as alcohol (Al). The formation of secondary amines is illustrated by way of example by the following reaction equation (1).

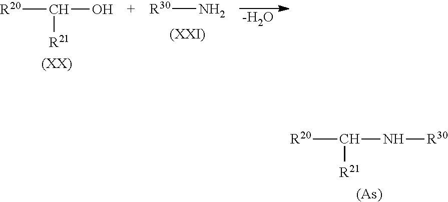

The present invention therefore also provides a process for preparing secondary amines (As) by alcohol amination of alcohols (Al), in which a primary or secondary monoalcohol is used as alcohol and a primary amine is used as aminating agent (Am).

The present invention further provides a process in which a primary or secondary monoalcohol is used as alcohol (Al) and a primary amine is used as aminating agent (Am) and a secondary amine (A) is obtained as amine (As).

Tertiary amines (At) are obtained when a primary or secondary monoalcohol is used as alcohol (Al) and a secondary amine is used as aminating agent (Am). Preference is given to using a primary alcohol as alcohol (Al). The formation of tertiary amines is illustrated by way of example by the following reaction equation (2).

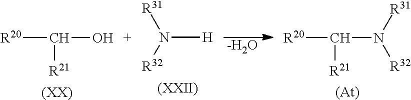

The present invention therefore also provides a process for preparing tertiary amines (At) by alcohol amination of alcohols (Al), in which a primary or secondary monoalcohol is used as alcohol and a secondary amine is used as aminating agent (Am).

The present invention further provides a process in which a primary or secondary monoalcohol is used as alcohol (Al) and a secondary amine is used as aminating agent (Am) and a tertiary amine (At) is obtained as amine (A).

Tertiary amines (At) can also be obtained when a primary or secondary monoalcohol is used in excess, preferably at least twice the molar amount, as alcohol (Al) and a primary amine is used as aminating agent (Am). Preference is given to using a primary alcohol as alcohol (Al). The formation of tertiary amines (At) is illustrated by way of example by the following reaction equation (3).

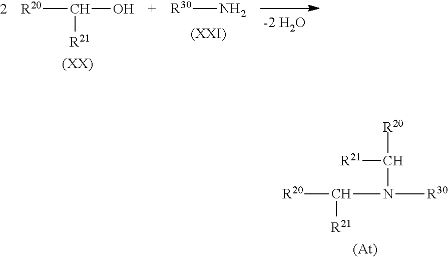

The present invention therefore also provides a process for preparing tertiary amines (At) by alcohol amination of alcohols (Al), in which a primary or secondary monoalcohol is used in excess, preferably at least twice the molar amount, as alcohol and a primary amine is used as aminating agent (Am).

The present invention further provides a process in which a primary or secondary monoalcohol is used in excess, preferably at least twice the molar amount, as alcohol (Al) and a secondary amine is used as aminating agent (Am) and a tertiary amine (At) is obtained as amine (A).

Tertiary amines (At) can also be obtained when a diol having primary or secondary hydroxyl groups is used as alcohol (Al) and a primary amine is used as aminating agent (Am). Preference is given to using a diol (XXa) comprising two primary hydroxyl groups as alcohol (Al). The formation of tertiary amines is illustrated by way of example by the following reaction equation (4).

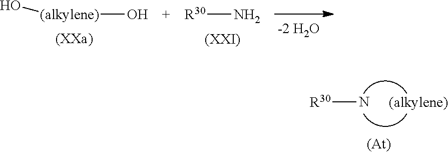

For the present purposes, the term “alkylene” refers to unsubstituted or at least monosubstituted divalent radicals. Preference is given to unsubstituted divalent radicals selected from the group consisting of ethylene, trimethylene, tetramethylene, pentamethylene, hexamethylene, heptamethylene and octamethylene.

The present invention therefore also provides a process for preparing tertiary amines (At) by alcohol amination of alcohols (Al), in which a diol having two primary hydroxyl groups is used as alcohol and a primary amine is uses as aminating agent (Am).

The present invention further provides a process in which a diol having two primary hydroxyl groups is used as alcohol (Al) and a primary amine is used as aminating agent (Am) and a tertiary amine (At) is obtained as amine (A).

The reaction outlet formed in the reaction generally comprises the corresponding amination product (i.e. a secondary or tertiary amine), any solvent used, the complex catalyst (I), any unreacted starting materials and the water or reaction formed.

### EXAMPLES

### Preparation of the Complex Catalyst (I)

[Cp*IrCl₂]₂ (79.6 mg, 0.1 mmol), amino acid (0.2 mmol) and K₂CO₃ (31.5 mg, 0.3 mmol) were suspended in 15 ml of dry acetonitrile (CH₃CN) under an argon atmosphere. The mixture was subsequently degassed by passing argon through the mixture via a cannula for 10 minutes. The mixture was subsequently stirred at room temperature for 24 hours. The mixture obtained in this way was concentrated under reduced pressure and the residue was taken up in dry dichloromethane (CH₂Cl₂; 10 ml). The suspension was filtered through Celite and the filtercake was washed a number of times with dichloromethane (total amount 50 ml). The combined yellow filtrates were concentrated to a volume of 5 ml and covered with an excess of dry pentane (30 ml). After 48 hours, the solvent mixture was decanted off and the crystals were washed with dry pentane. The complex catalyst (I) obtained in this way was dried under reduced pressure to give a yellow or orange solid.

The formation of the complex catalyst is shown by the following reaction equation (5)

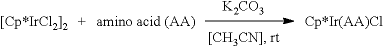

The complex catalysts Ia, Ib, Ic and Id were synthesized; “Gly” is L-glycine. “Val” is L-valine. “Sar” is L-sarcosine and “Pro” is L-proline; “d.r” indicates the diastereomer ratio, “rt” means room temperature.

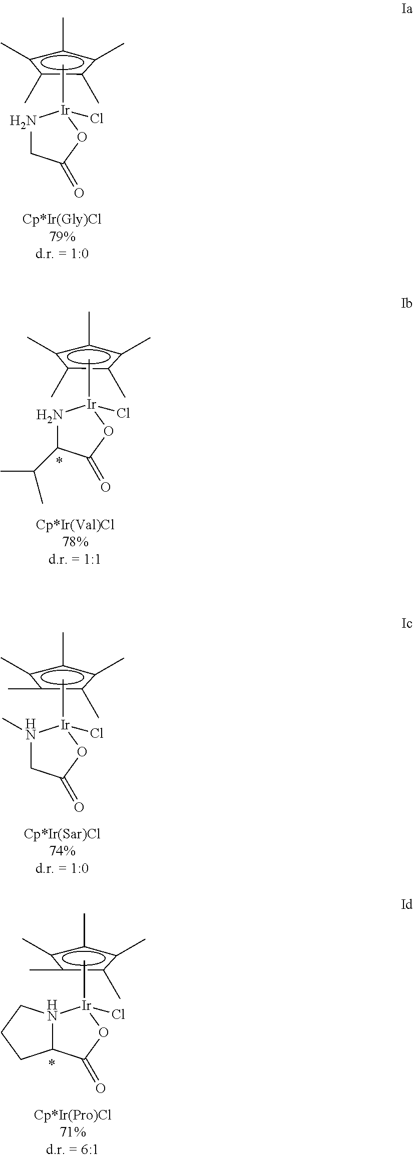

The analytical characterization of the complex catalysts Ia, Ib, Ic and Id is reported below.

### Cp*Ir(Gly)Cl (Ia):

¹H-NMR (MeOD, 500 MHz): δ=1.70 (s, 15H, 5×CH₃), 3.40 (dd, 2H, CH₂, J=15.6 Hz, J=15.3 Hz),

¹³C-NMR (MeOD, 125.7 MHz): δ=9.1 (5×CH₃), 45.9 (CH₂), 85.6 (5×C_{q}), 186.9 (C_{q}).

MS (FAB): m/e=438.6.

Elemental analysis: calc. C, 32.98%, H, 4.38%, N, 3.21%. found C, 31.49%, H, 4.32%, N, 2.97%.

### Cp*Ir(Val)Cl (Ib):

¹H-NMR (MeOD, 500 MHz): δ=0.91 (dd, 3H, CH₃, J=6.8 Hz, J=25.1 Hz), 1.07 (m, 3H, CH₃), 1.71 (s, 15H, 5×CH₃), 2.29 (m, 1H, CH), 3.25 (m, 1H, CH),

¹³C-NMR (MeOD, 125.7 MHz): δ=9.1 (5×CH₃), 17.0 (CH₃), 19.3 (CH₃), 32.4 (CH), 61.8 (CH), 86.5 (5×C_{q}), 184.4 (C_{q}).

MS (FAB): m/e=480.2.

Elemental analysis: calc. C, 37.61%, H, 5.26%, N, 2.92%. found 37.05%, H, 5.40%, N, 2.88%.

### Cp*Ir(Sar)Cl (Ic):

¹H-NMR (MeOD, 500 MHz): δ=1.67 (s, 15H, 5×CH₃), 2.78 (s, 3H, CH₃), 3.39 (dd, 2H, CH₂, J=14.8 Hz, J=61.8 Hz),

¹³C-NMR (MeOD, 125.7 MHz): δ=9.3 (5×CH₃), 40.8 (CH₂), 57.0 (CH₃), 86.2 (5×C_{q}), 185.7 (C_{q}).

MS (FAB): m/e=452.1.

Elemental analysis: calc. C, 34.62%, H, 4.69%, N, 3.11%. found C, 33.50%, H, 4.50%, N, 2.92%.

### Cp*Ir(Pro)Cl (Id):

¹H-NMR (MeOD, 500 MHz): δ=1.70 (s, 15H, 5×CH₃), 1.78-1.97 (m, 4H, 2×CH₂), 2.19 (m, 2H, CH₂), 2.89 (m, 1H, CH₂), 3.64 (m, 1H, CH₂), 3.92 (m, 1H, CH),

¹³C-NMR (MeOD, 125.7 MHz): δ=9.4 (5×CH₃), 27.9 (CH₂), 30.2 (CH₂), 56.3 (CH₂), 64.0 (CH), 85.8 (5×C_{q}), 188.4 (C_{q}).

MS (FAB): m/e=478.2.

Elemental analysis: calc. C, 37.77%, H, 4.86%, N, 2.94%. found C, 38.52%, H, 5.15%, N, 2.83%.

### Alcohol Amination of 1-Octylamine (1) Using 1-Hexanol (2)

In an ACE pressure tube, 1-hexanol (1.0 mmol, 102.2 mg), 1-octylamine (1.0 mmol, 128.2 mg) and the respective complex catalyst (2 mol %) were dissolved in 0.5 ml of dry toluene (if the reaction was carried out in water, 0.1 ml of water were used instead of the toluene—see table 7) under an argon atmosphere. The reaction vessel was closed by means of a Teflon stopper and heated at the temperature indicated for 24 hours while stirring. The mixture was subsequently cooled to room temperature and diluted with water (10 ml). The crude product was extracted with dichloromethane (2×10 ml). The combined organic phases were washed with a saturated sodium hydrogencarbonate solution and a saturated sodium chloride solution and dried over sodium sulfate (Na₂SO₄). After filtration, the solvent was distilled off under reduced pressure and the resulting product of the alcohol amination was purified by column chromatography over Florisil (magnesium silicate) using a mixture of dichloromethane and methanol in a ratio of from 30:1 to 10:1. After removal of the solvent, a colorless liquid was obtained.

The reaction obeys the reaction equation (6) (not depicted stoichiometrically)

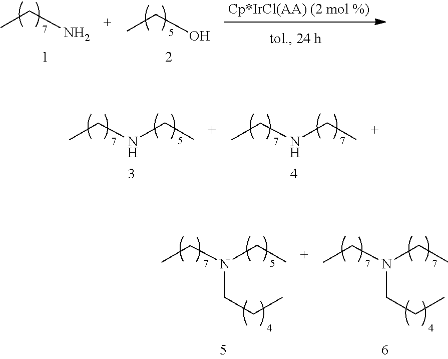

The results are shown in table 1 below.

TABLE 1

| Ex- | | | Con- | Selectivity | | | |
|---|---|---|---|---|---|---|---|
| am- | | Temp. | version | 3 | 4 | 5 | 6 |
| ple | Complex catalyst | [° C.] | (%) | (%) | (%) | (%) | (%) |
| 1 | Cp*Ir(Gly)Cl (Ia) | 125 | 100 | 53 | 12 | 21 | 14 |
| 2 | Cp*Ir(Val)Cl (Ib) | 125 | 100 | 59 | 12 | 14 | 15 |
| 3 | Cp*Ir(Sar)Cl (Ic) | 125 | 100 | 44 | 20 | 14 | 22 |
| 4 | Cp*Ir(Pro)Cl (Id) | 125 | 100 | 40 | 15 | 16 | 29 |
| 5 | Cp*Ir(Gly)Cl (Ia) | 105 | 100 | 82 | 12 | 2 | 4 |
| 6 | Cp*Ir(Val)Cl (Ib) | 105 | 100 | 95 | 5 | 0 | 0 |
| 7 | Cp*Ir(Sar)Cl (Ic) | 105 | 100 | 89 | 11 | 0 | 0 |
| 8 | Cp*Ir(Pro)Cl (Id) | 105 | 100 | 50 | 13 | 17 | 20 |
| 9 | Cp*Ir(Gly)Cl (Ia) | 95 | 100 | 94 | 3 | 3 | 0 |
| 10 | Cp*Ir(Val)Cl (Ib) | 95 | 93 | 94 | 6 | 0 | 0 |
| 11 | Cp*Ir(Sar)Cl (Ic) | 95 | 100 | 97 | 3 | <1 | 0 |
| 12 | Cp*Ir(Pro)Cl (Id) | 95 | 100 | 86 | 8 | 6 | 0 |
| 13 | Glycine | 95 | 0 | 0 | 0 | 0 | 0 |
| 14 | [Cp*IrCl₂]₂ | 95 | 29 | 19 | 0 | 0 | 0 |

All the complex catalysts Ia, Ib, Ic and Id tested display good catalytic activity at temperatures of 125° C. and 105° C. At these temperatures, complete conversion of the starting materials used was achieved in all cases. However, in addition to the formation of the desired target product N-hexyloctylamine (3), the formation of the secondary amine (4) and the tertiary amines (5 and 6) was also observed. Reducing the temperature to 95° C. improved the selectivity of the formation of the desired target product (3) significantly (see examples 9 to 12 in table 1).

When an amino acid (glycine; see example 13 in table 1) was used, no conversion was observed. In example 15 in table 1, the reaction was carried out in the presence of [Cp*IrCl₂]₂ at 95° C. Here, only very low conversions of the starting materials and unsatisfactory selectivity to the target product N-hexyloctylamine (3) were achieved.

The target product N-hexyloctylamine (3) obtained was characterized by analysis. The results are shown below.

¹H-NMR (CDCl₃, 200 MHz): δ=0.86 (t, 6H, 2×CH₃, J=6.1 Hz), 1.26 (m, 16H, 8×CH₂), 1.60 (m, 4H, 2×CH₂), 2.66 (t, 4H, 2×CH₂, J=7.4 Hz), 4.07 (bs, 1H, NH),

¹³C-NMR (CDCl₃, 50.3 MHz): S=14.0 (CH₃), 14.0 (CH₃), 22.5 (CH₂), 22.6 (CH₂), 26.9 (CH₂), 27.3 (CH₂), 28.9 (CH₂), 29.0 (CH₂), 29.2 (CH₂), 29.4 (CH₂), 31.6 (CH₂), 31.8 (CH₂), 49.5 (2×CH₂).

MS (EI): m/e (%): 213 (9), 142 (98), 114 (100), 100 (3), 84 (4), 70 (7), 57 (12).

HR-MS: calculated: 213.2457 found: 213.2438.

To examine the reactivity of the complex catalysts Ia, Ic and Id, the alcohol amination was carried out at 95° C. and stopped after a reaction time of 6 hours. These reaction conditions gave a reaction mixture corresponding to reaction equation (7) below.

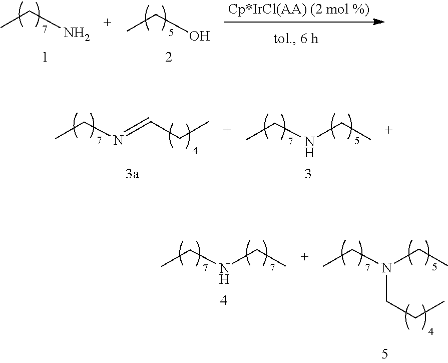

The results are shown in table 2 below.

TABLE 2

| Ex- | | | Con- | Selectivity | | | |
|---|---|---|---|---|---|---|---|
| am- | | Temp. | version | 3a | 3 | 4 | 5 |
| ple | Complex catalyst | [° C.] | (%) | (%) | (%) | (%) | (%) |
| 15 | Cp*Ir(Gly)Cl (Ia) | 95 | 34 | 19 | 79 | 2 | <1 |
| 16 | Cp*Ir(Sar)Cl (Ic) | 95 | 65 | 9 | 90 | 0 | 1 |
| 17 | Cp*Ir(Pro)Cl (Id) | 95 | 90 | 8 | 82 | 7 | 3 |

After 6 hours, the intermediate (3a) could be detected. At a reaction time of 6 hours, conversions in the range from 34 to 90% were achieved. The selectivity to the target product (3) was in the range from 79 to 90%.

Preparation of Secondary Amines (As) from Aniline Derivatives as Aminating Agent (Am)

The aniline derivatives shown in table 3 were reacted with 1-hexanol. The reaction was carried out according to the above-described method. Toluene was used as solvent and Cp*Ir(Pro)Cl (2 mol %) was used as catalyst. The reaction was carried out in toluene at 95° C. for 24 hours. The reaction obeys the general reaction equation (8).

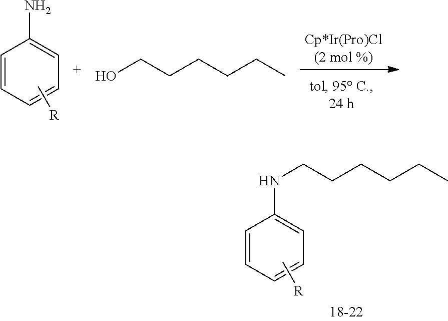

TABLE 3

| Example | R | Yield (%) |
|---|---|---|
| 18 | H | 98 |
| 19 | p-OMe | 99 |
| 20 | p-Cl | 85 |
| 21 | o,p-Me | 83 |
| 22 | p-CO₂Me | 86 |

The desired target products (18 to 22) were obtained in good yields. The formation of N-dialkylated aniline derivatives was not observed.

Aniline derivatives were reacted with benzyl alcohol in a manner analogous to the above-described reaction. The reaction in this case occurred according to reaction equation (9) below.

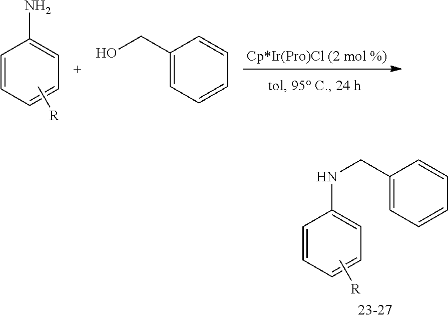

The results are shown in table (4). Here too, good yields of the target products (23 to 27) were obtained.

TABLE 4

| Example | R | Yield (%) |
|---|---|---|
| 23 | H | 97 |
| 24 | p-OMe | 100 |
| 25 | p-Cl | 95 |
| 26 | o,p-Me | 81 |
| 27 | p-CO₂Me | 82 |

In addition, aniline was reacted with various benzyl alcohol derivatives. The reaction here obeys the following reaction equation (10)

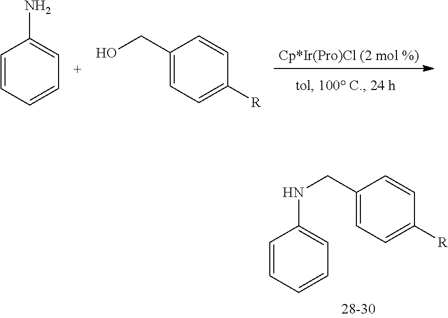

The results are shown in table 5.

TABLE 5

| Example | R | Yield (%) |
|---|---|---|
| 28 | OMe | 93 |
| 29 | Cl | 90 |
| 30 | CO₂Me | 96 |

The reaction was in this case carried out at 100° C. The secondary amines (As) were obtained in yields of >90%.

The reaction of further alcohols (Al) with further aminating agents (Am) is shown in table (6) below. The reaction was carried out in toluene as solvent in the presence of 2 mol % of Cp*Ir(Pro)Cl as catalyst. The reaction conditions and yields are shown in table (6) below.

TABLE 6

| | Aminating agent | T | Reaction | | Yield |
|---|---|---|---|---|---|
| Alcohol (Al) | (Am) | [° C.] | time [h] | Product | (%) |
| 1-Hexanol | 1-Pentylamine | 95 | 24 | 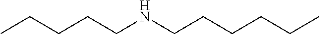 | 92 |
| 1-Hexanol | 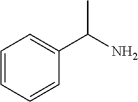 | 130 | 36 | 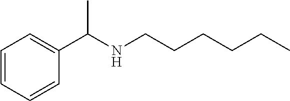 | 91 |
| Benzyl alcohol | Cyclohexylamine | 150 | 72 | 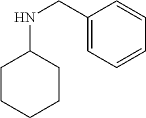 [SMILES: c1ccc(CNC2CCCCC2)cc1] | 40 |
| 1-Hexanol | 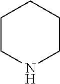 | 126 | 36 | 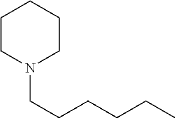 | 84 |
| Cyclo- hexanol | 1-Octylamine | 115 | 24 | 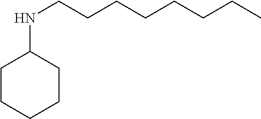 | 89 |
| 1,4-Butane- diol | Benzylamine | 95 | 24 | 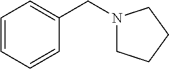 [SMILES: c1ccc(CN2CCCC2)cc1] | 94 |
| 1,6-Hexane- diol | Benzylamine | 95 | 24 | 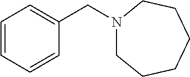 [SMILES: c1ccc(CN2CCCCCC2)cc1] | 91 |
| 1-Propanol | Ethylamine | 100 | 24 | 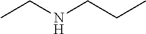 | >90 |
| 1-Hexanol | 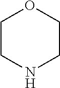 | 130 | 24 | 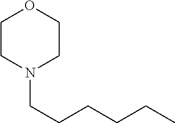 | 87 |
| 1-Hexanol | 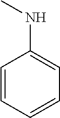 | 130 | 24 | 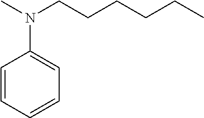 | 93 |

The results of the alcohol amination in water as solvent are shown in table 7.

TABLE 7

| | Aminating | T | Reaction | | Yield |
|---|---|---|---|---|---|
| Alcohol (Al) | agent (Am) | [° C.] | time [h] | Product | (%) |
| 1-Hexanol | 1-Octyl-amine | 100 | 24 | 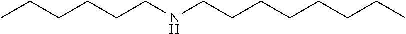 | 98 |
| 1,6-Hexane- diol | Benzylamine | 100 | 24 | 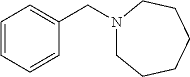 [SMILES: c1ccc(CN2CCCCCC2)cc1] | 96 |
| Benzyl alcohol | Aniline | 100 | 24 | 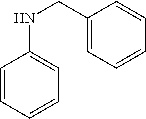 [SMILES: c1ccc(CNc2ccccc2)cc1] | 94 |

Selectivities and yields were determined either by isolation of the product or by means of GC using the internal standard biphenyl.

The analysis of the reaction mixtures by GC-MS were carried out on an Agilent 19091S-433 modular GC using a capillary injection system in the split mode and a flame ionization detector. A standard HP-5 capillary column (Agilent 19091S-433, 5% phenylmethylsiloxane, capillary 30 m×250 μm×0.25 μm) was used (helium flow 1.0 ml/min, temperature program: initial 50° C. for 2 min, 10° C./min to 280° C., 280° C. for 2 min).

1. A process for preparing an amine by amination of an alcohol, the process comprising aminating an alcohol with an aminating agent in the presence of a complex catalyst comprising iridium and an amino acid, to form an amine and with elimination of water.

2. The process according to claim 1, wherein the complex catalyst comprises an α-amino acid.

3. The process according to claim 1, wherein the process is homogeneously catalyzed.

4. The process according to claim 1, wherein the process occurs in the presence of a complex catalyst of formula (I): 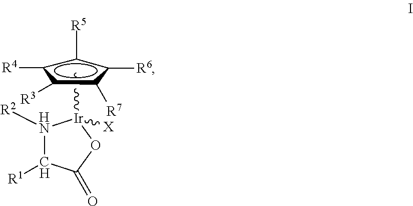
wherein:
R¹ and R² independently represent hydrogen, unsubstituted or at least monosubstituted C₁-C₁₀-alkyl, C₅-C₁₀-cycloalkyl, C₅-C₁₀-heterocyclyl, C₅-C₁₀-aryl or C₅-C₁₀-heteroaryl,
or
R¹ and R² together with the atoms to which they are bound form an unsubstituted or at least monosubstituted five- to ten-membered ring system,
where the optional substituents are selected from the group consisting of NR⁸R⁹, OR¹⁰, SR¹¹, C(O)OR¹², C(O)NR¹³R¹⁴, NHC(NH₂)₂⁺ and unsubstituted or at least monosubstituted C₅-C₁₀-aryl and C₅-C₁₀-heteroaryl, where the optional substituents are selected from the group consisting of OH and NH₂;
X represents fluoride, chloride, bromide or iodide;
R³, R⁴, R⁵, R⁶ and R⁷ each independently represent hydrogen, methyl, ethyl, n-propyl, isopropyl or phenyl; and
R⁸, R⁹, R¹⁰, R¹¹, R¹², R¹³ and R¹⁴ each independently represent hydrogen or C₁-C₆-alkyl.

5. The process according to claim 4, wherein:
R³, R⁴, R⁵, R⁶ and R⁷ each represent methyl; and
X represents chloride.

6. The process according to claim 1, wherein the amino acid is selected from the group consisting of alanine, valine, leucine, isoleucine, proline, tryptophan, phenylalanine, methonine, glycine, serine, tyrosine, threonine, cysteine, asparagine, glutamine, aspartate, glutamate, lysine, arginine, histidine, citrulline, homocysteine, homoserine, (4R)-4-hydroxyproline, (5R)-5-hydroxylysine, ornithine and sarcosine.

7. The process according to claim 1, wherein the process occurs at temperatures in the range from 50° C. to 150° C.

8. The process according to claim 1, wherein the process occurs without the addition of a base selected from the group consisting of an alkali metal hydroxides, an alkaline earth metal hydroxide, an alkali metal alkoxide, an alkaline earth metal alkoxide, an alkali metal carbonate, an alkaline earth metal carbonate, an alkali metal hydrogencarbonate and an alkaline earth metal hydrogencarbonate.

9. The process according to claim 1, wherein the process occurs in the presence of a solvent.

10. The process according to claim 9, wherein the solvent is a nonpolar solvent selected from the group consisting of toluene, xylene and mesitylene.

11. The process according to claim 9, wherein the solvent is a polar solvent selected from the group consisting of water, dimethylformamide, formamide, tert-amyl alcohol and acetonitrile.

12. The process according to claim 1, wherein:
the alcohol is a primary or secondary monoalcohol;
the aminating agent is a primary amine; and
the amine is a secondary (As) is obtained as amine (A).

13. The process according to claim 1, wherein:
the alcohol is a primary or secondary monoalcohol;
the aminating agent is a secondary amine; and
the amine is a tertiary amine.

14. The process according to claim 1, wherein:
the alcohol is a diol having two primary hydroxyl groups;
the aminating agent is a primary amine; and
the amine is a tertiary amine.
